(19) United States
(12) Patent Application Publication
(10) Pub. No.: US 2025/0230122 A1
(43) Pub. Date: Jul. 17, 2025

(54) TEMPERATURE CONTROL IN THE PREPARATION OF ALICYCLIC POLYCARBOXYLIC ACIDS AND ESTERS THEREOF

(71) Applicant: Evonik Oxeno GmbH & Co. KG, Marl (DE)

(72) Inventors: Grzegorz Ziomek, Recklinghausen (DE); Thomas Schneider, Schermbeck (DE); Michael Grass, Haltern am See (DE); Berno Simon, Marl (DE)

(73) Assignee: Evonik Oxeno GmbH & Co. KG, Marl (DE)

(21) Appl. No.: 19/022,100

(22) Filed: Jan. 15, 2025

(30) Foreign Application Priority Data
Jan. 16, 2024 (EP) 24152110.3

(51) Int. Cl. C07C 67/303; B01J 8/04; C07C 67/48

(52) U.S. Cl. CPC C07C 67/303; B01J 8/0492; B01J 8/0496; C07C 67/48; B01J 2208/00176; C07C 2601/14

(57) ABSTRACT

In the technical field of the preparation of alicyclic compounds, a ring hydrogenation of aromatic compounds prepares alicyclic compounds in a process for preparing alicyclic compounds. The alicyclic compounds may preferably be alicyclic carboxylic acids and esters thereof. An apparatus for carrying out this process may further be provided.

### CROSS-REFERENCE TO RELATED APPLICATIONS

This patent application claims priority to European Patent Application No. 24152110.3, filed on Jan. 16, 2024, in the European Patent Office, the entire disclosure of which is hereby incorporated by reference herein.

### BACKGROUND OF THE INVENTION

### Field of the Invention

The invention is in the technical field of the preparation of alicyclic compounds by ring hydrogenation of aromatic compounds. The invention provides processes for preparing alicyclic compounds, preferably alicyclic carboxylic acids and esters thereof, and apparatuses for carrying out this process.

### Description of Related Art

Alicyclic polycarboxylic esters, such as for example the esters of cyclohexane-1,2-dicarboxylic acid, are used as lubricating oil component and as aids in metal processing. They are also used as plasticizers for polyolefins and for PVC.

For the plasticizing of PVC, esters of phthalic acid, such as for example dinonyl or didecyl esters, are predominantly used. The use of these phthalates is becoming increasingly controversial with the public and the use in plastics might be restricted. Alicyclic polycarboxylic esters, of which some have already been described in the literature as plasticizers for plastics, can be a suitable choice for possible substitutes for the plasticizers that are subject to restrictions.

In most cases, the most economical route for the preparation of alicyclic polycarboxylic esters is the ring hydrogenation of the corresponding aromatic polycarboxylic esters, for example the above-mentioned phthalates. For this, a number of processes are already known:

In DE 102 32 868.4 and DE 102 25 565.2, the hydrogenation of the aromatic polycarboxylic esters to give the corresponding alicyclic polycarboxylic esters is carried out in two reactors connected in series, the first being operated in loop mode (partial recycling of the reactor output) and the second being operated in straight pass mode. The first loop reactor can also be replaced by a plurality of small loop reactors connected in series or in parallel, these reactors having a common circuit.

Application EP 1 676 829 discloses a process for continuous catalytic hydrogenation in at least two reactors connected in series, wherein the catalyst volume is kept as low as possible. The first reactor is operated in loop mode and at least one further reactor is operated in straight pass mode. Since the hydrogenation is an exothermic process, a fairly high temperature is achieved in the first reactor, which is operated in loop mode, at the start of such a process with fresh catalyst. The activity of the catalyst decreases over the time of the continuous hydrogenation. The decrease in the activity of the catalyst in the first reactor is then typically compensated by increasing the temperature in the first reactor. However, increasing the temperature in the first reactor may lead to problems in the second reactor, which is operated in straight pass mode. On account of safety-relevant aspects, the temperature in the first and in the second reactor cannot become infinitely high. This may lead to the first reactor no longer being able to be operated at a sufficiently high temperature, since otherwise there is a possible threat of temperature-induced shutdown of the first and in particular of the second reactor on account of safety aspects. Premature shutdown of the reactor may lead to a reduced yield and to insufficient hydrogenation of the reactant in the product stream from the second reactor.

### SUMMARY OF THE INVENTION

The primary object of the present invention was therefore to provide a process for the catalytic hydrogenation of aromatic compounds, preferably of aromatic polycarboxylic acids and esters thereof, which has a high yield and in which the product stream comprises a consistently high proportion of alicyclic compounds as a product of the hydrogenation of the aromatic compounds used.

### BRIEF DESCRIPTION OF THE DRAWINGS

The FIGURE shows an apparatus for preparing one or more alicyclic compounds.

### DETAILED DESCRIPTION OF THE INVENTION

This primary object has been achieved by provision of a process for preparing one or more alicyclic compounds, comprising the steps of:
i. providing a stream A (2b) comprising one or more aromatic compounds and a hydrogen-containing hydrogenation gas;
ii. feeding stream A (2b) having a temperature T₁ to a first hydrogenation unit (1) and hydrogenating the one or more aromatic compounds to give one or more corresponding alicyclic compounds;
iii. obtaining a mixture as first product stream (3) having a temperature T₂ comprising aromatic compounds and alicyclic compounds;
iv. separating the product stream (3) obtained in step iii. into a substream (8), which is fed to stream A (2b) in step i. and hydrogenated again as part of stream A (2b) in step ii., and a substream (6c), which is fed as stream B (9) at a temperature T₃ into one or more further hydrogenation units (11);
v. hydrogenating the aromatic compounds present in stream B (9) to give the corresponding alicyclic compounds in the one or more further hydrogenation units (11), and
vi. obtaining a product stream (12) having a temperature T₄ comprising one or more alicyclic compounds corresponding to the one or more aromatic compounds provided in step i,
   wherein at least temporarily the temperature T₁ is unequal to the temperature T₃, preferably wherein the temperature T₁ is higher than the temperature T₃.

“Alicyclic compounds” in the context of the present invention are understood to mean those compounds which have a saturated ring system with an aliphatic structure. Such compounds are also known as cycloaliphatic compounds. Preferably, the alicyclic compounds that are obtained as products in the context of the present invention have a cyclohexane ring.

“Aromatic compounds” in the context of the present invention are understood to mean those compounds which have at least one ring system which, according to Huckel's rule contains a number of 4n+2 delocalized electrons in conjugated double bonds, free electron pairs or unoccupied p-orbitals. Preferably, the aromatic compounds that are used as starting materials in the context of the present invention have a benzene ring.

A “hydrogen-containing hydrogenation gas” is a gas containing hydrogen. In the context of the reaction on which the present invention is based, hydrogen is used as a further reactant in addition to the aromatic compounds. In the hydrogenation reaction carried out, the double bonds in the ring of the aromatic compounds used, preferably of the benzene ring, are hydrogenated by an addition reaction of the hydrogen and thereby broken. The reaction takes place in the presence of a solid catalyst. The hydrogen molecule in the hydrogen-containing hydrogenation gas intermediately bonds to the metal atom of the catalyst and the bond between the two hydrogen atoms in the hydrogen molecule is weakened and can interact with an electron-rich multiple bond (double bond). The hydrogenation takes place when two hydrogen atoms are formally transferred to a double bond. This breaks the double bonds in the aromatic compounds and alicyclic compounds are obtained.

The hydrogenation gases used can be any hydrogen-containing gas mixtures which do not contain any harmful amounts of catalyst poisons such as carbon monoxide or hydrogen sulfide. The use of inert gases is optional, preference being given to using hydrogen in a purity of greater than 95%, in particular greater than 98%. Inert gas fractions may be, for example, nitrogen or methane. It is preferable for such an amount of hydrogen to be present in the hydrogenation units that this hydrogen is present in excess, in particular in an excess of 1% to 200%, preferably in an excess of 3% to 100% and particularly preferably in an excess of 5% to 50%, based on the stoichiometric amount needed to achieve the desired conversion or the conversion that is possible in the hydrogenation unit. Setting a sufficient excess of hydrogen can have advantageous effects on the complete hydrogenation of the aromatic bonds.

A “hydrogenation unit” in the context of the present invention is to be understood as meaning a hydrogenation reactor or a plurality of reactors connected in series or a plurality of reactors connected in parallel with one another, or a reactor group consisting of reactors connected in parallel and in series. It is therefore to be understood as meaning a reactor or a reactor arrangement that can perform the function of a reactor in the process according to the invention.

The individual hydrogenation units can be charged with fresh hydrogen. However, in order to minimize the hydrogen consumption and the effluent losses resulting from the offgas, it is expedient to use the offgas of one hydrogenation unit as hydrogenation gas for another or the same hydrogenation unit. In addition, the offgas of one hydrogenation unit can be reused as fresh hydrogen after work up. For example, in a process that is carried out in two series-connected hydrogenation units each having a hydrogenation unit, it is advantageous to feed fresh hydrogen into the first hydrogenation unit and to conduct the offgas of the first hydrogenation unit into the second hydrogenation unit. In this case, starting material and hydrogenation gas flow through the hydrogenation units in opposite sequence. It is advantageous in this process regime to keep the hydrogen excess, based on the stoichiometrically required amount, below 30%, in particular below 20%.

In the context of the present invention, “recycling” or “loop mode” is understood to mean the at least partial recycling of the product stream from a hydrogenation unit as part of the input stream to the same hydrogenation unit. The product stream or the mixture obtained from a hydrogenation unit is split in the process. This means that a substream of the hydrogenation effluent or product stream from the first hydrogenation unit is passed into the first hydrogenation unit together with fresh reactant as stream A. The other substream of the hydrogenation output or product stream of the first hydrogenation unit is hydrogenated in a second hydrogenation unit which is preferably operated in straight pass mode. Instead of one large hydrogenation unit in loop mode, it is also possible to use two or more smaller units that are arranged in series or in parallel. Instead of one large hydrogenation unit with straight pass flow, it is likewise possible to operate two or more series- or parallel-connected units. However, preference is given to using just one hydrogenation unit operated in loop mode and one unit operated in straight pass mode. The process can also be carried out in tube bundle reactors.

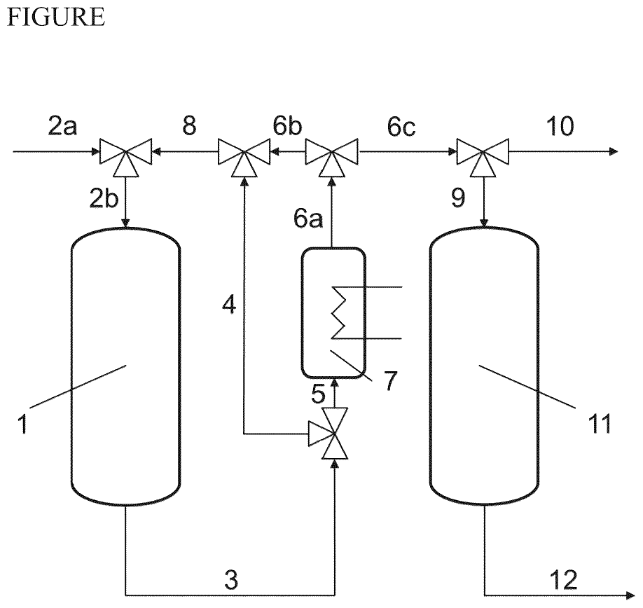

In the example of FIG. 1, this means that the product stream (3) can be fed via the pipelines (4, 8) together with fresh reactant (2a) as input stream or stream A (2b) to the hydrogenation unit (1).

It is preferred in the context of the process according to the invention when step iv. is carried out with a circulation ratio of 1:10 to 1:50, preferably of 1:10 to 1:40, particularly preferably of 1:30. This ratio means, with the value 1:10, for example, that ten tonnes of the product stream are supplied back to the top of the first hydrogenation unit and one tonne is supplied to the at least one further hydrogenation unit.

The circulation ratio is preferably set such that an overall conversion of 80% to 99%, preferably of 85% to 97%, is achieved in the first of the series-connected hydrogenation units and a conversion of 80% to 100%, preferably of 85% to 100% is achieved in the second hydrogenation unit, based on the starting concentration of the compound to be hydrogenated at the inlet of the respective hydrogenation unit. If three or more hydrogenation units are used, the conversions must be adjusted accordingly.

The at least one further hydrogenation unit can likewise be operated in loop mode or in straight pass mode, which means that there is no recycling of the recyclate into the same hydrogenation unit.

The hydrogenation can be conducted in the absence or preferably in the presence of a solvent. Solvents used may be all liquids that form a homogeneous solution with the starting material and product, are inert under hydrogenation conditions and can be easily separated off from the product. The solvent may also be a mixture of two or more substances and may optionally contain water.

For example, it is possible to use the following substances as solvent: straight-chain or cyclic ethers such as tetrahydrofuran or dioxane and also aliphatic alcohols in which the alkyl radical has 1 to 13 carbon atoms.

Alcohols usable with preference are isopropanol, n-butanol, isobutanol, n-pentanol, 2-ethylhexanol, nonanols, industrial nonanol mixtures, decanol, industrial decanol mixtures, tridecanols.

When alcohols are used as solvent, it may be expedient to use that alcohol or that alcohol mixture that would form in the hydrolysis of the product. This would rule out by-product formation through transesterification. A further preferred solvent is the hydrogenation product itself.

The use of a solvent allows the concentration of aromatic compounds in the reactor feed to be limited, as a result of which better temperature control in the reactor can be achieved. This can minimize side reactions and accordingly bring about an increase in product yield. Preferably, the concentration of aromatic compounds in the reactor feed may be between 1% and 35% by weight, especially between 5% and 25% by weight, based on the total amount of the reactant used. The desired concentration range in the case of reactors that are operated in loop mode can be adjusted via the circulation ratio (quantitative ratio of recycled hydrogenation output to starting material).

The process according to the invention will be described by way of example below using the example of the system shown in FIG. 1.

In step i., at least one aromatic compound and a hydrogen-containing hydrogenation gas are provided as reactants and passed via the input stream as stream A (2b) into a first hydrogenation unit (1). In this case, fresh reactant is fed in the input stream (2a) and passed to the first hydrogenation unit via the input stream (2b). The input stream (2b) has the temperature T₁. In this first hydrogenation unit (1), the hydrogenation then takes place in step ii. and, in step iii., at the end of the hydrogenation unit, a mixture is obtained as product stream (3) with hydrogenated compounds (alicyclic compounds) and unhydrogenated compounds (aromatic compounds). This mixture or the product stream (3) has the temperature T₂ and is then divided into two substreams in step iv. with the one substream being passed as stream (8) to stream A (2b) and being subjected to a renewed hydrogenation according to step ii. with fresh reactant (2a) in the first hydrogenation unit (1). The second substream, stream B (6c), is introduced via the input stream (9) into a second hydrogenation unit (11) and has the temperature T₃. It is hydrogenated in at least one further hydrogenation unit (11) in step v., so that the reactant which has not been converted in the first hydrogenation unit (1) is hydrogenated in this second hydrogenation unit (11) to give the corresponding alicyclic compounds. The product mixture obtained as stream (12) in step vi. has the temperature T₄. The division of the product streams in step iv. can be effected either via a bypass as stream (4) or via a cooling device (7), preferably via a heat exchanger.

The product stream (12) at the outlet of the at least one further hydrogenation unit preferably comprises less than 0.3% by mass, preferably less than 0.1% by mass, especially less than 0.05% by mass, particularly preferably 0.005% by mass, of alicyclic compounds used as reactant.

The process parameters, such as product, by-product and reactant concentration and also temperature, are determined preferably by online analysis. Online analysis acquires the respective parameters in real time, preferably in the product output streams from the first (3) and/or each further hydrogenation unit (12). Preference is given to using a measurement method selected from the group consisting of reaction calorimeter, ATR-FT-IR spectroscopy, RAMAN spectroscopy, IR spectroscopy, UV and/or UV-VIS spectroscopy or combinations thereof. On the basis of the process parameters thus determined, the temperature T₁ could be set in a targeted manner after previous determination of a limit value. This setting can also be effected in automated fashion, i.e. with the aid of computer technology.

It is preferred in the context of the present invention that the hydrogenation of the aromatic compounds provided in step i. is effected with the hydrogen-containing gas provided in step i. over one or more solid catalysts arranged in a fixed bed of the hydrogenation units.

It is further preferred that the solid catalyst includes at least one metal from the eighth transition group of the periodic table of the elements. Preference is given to using, as active metals, platinum, rhodium, palladium, cobalt, nickel or ruthenium or a mixture of two or more thereof, with ruthenium being used in particular as active metal.

In addition to the metals already mentioned, at least one metal of transition group one and/or seven of the Periodic Table of the Elements can additionally be present in the catalysts. Preference is given to using rhenium and/or copper in addition to the metal from the eighth transition group of the periodic table of the elements.

The catalysts used in the context of this process are preferably metals, as defined above, applied to a support material. The support materials used are preferably materials containing micropores (pore diameter less than 2 nm), mesopores (pore diameter of 2 to 50 nm) and macropores (pore diameter greater than 50 nm). For instance, with respect to the pore type, support materials having the following pore combinations are usable:
a) mesopores only,
b) micropores and mesopores,
c) mesopores and macropores,
d) micropores and mesopores and macropores,
e) micropores and macropores.

Preference is given to using, as support materials, activated carbon, silicon carbide, aluminium oxide, silicon oxide, aluminosilicate, titanium dioxide, zirconium dioxide, magnesium oxide and/or zinc oxide, or mixtures thereof.

Preferably used as support materials are solids that are largely inert under hydrogenation conditions. Examples of these are activated carbon, silicon carbide, silicon dioxide, titanium dioxide and/or zirconium dioxide, and mixtures of these compounds. Very particular preference is given to using titanium dioxides as support materials. Titanium dioxide occurs in three polymorphs (anatase, rutile and brookite), of which anatase and rutile are the most common. A preferred support material is Aerolyst 7711® (Evonik Operations GmbH). This support material consists to an extent of 15% to 20% by mass of rutile and 80% to 85% by mass of anatase. Further examples of suitable titanium dioxide support materials are those produced on the basis of titanium oxides from a sulfuric acid process. They generally contain>98% anatase.

It is particularly preferred that the solid catalyst used for the hydrogenation in step ii. and/or v. is a catalyst comprising ruthenium as sole metal and titanium dioxide as support material. In a preferred embodiment, the same catalyst is used for the hydrogenation in step ii. and in step v.; particularly preferably, this is a catalyst comprising ruthenium as sole metal and titanium dioxide as support material.

In the process according to the invention, the hydrogenation in step ii. and/or v. is carried out in the liquid phase or in the gas phase. The hydrogenation can be conducted continuously or batchwise over suspended catalysts or those arranged in piece form in a fixed bed. In the process according to the invention, preference is given to continuous hydrogenation over a catalyst in fixed bed form, in which the product/starting material phase is mainly in the liquid state under the reaction conditions.

It is preferred that the hydrogenation in step ii. and/or v. is carried out at a pressure of 3 to 300 bar, preferably 15 to 200 bar, particularly preferably 50 to 150 bar.

It is further preferable that the hydrogenation in step ii. and/or iv. is carried out at a temperature of 50° C. to 250° C., preferably 70 to 200° C. This temperature is present after hydrogenation has been effected in the output streams (T₂ and T₄) of the hydrogenation units. Due to the exothermicity of the hydrogenation reaction, the reaction does not take place at a fixed temperature, but rather within a temperature range as described herein. The temperature of the reaction mixture thus rises as it flows through the hydrogenation unit. The temperature T₁ is therefore always lower than T₂ and the temperature T₃ is always lower than T₄.

According to the invention, it is provided that the temperature T₁ is at least temporarily unequal to the temperature T₃, preferably wherein the temperature T₁ is higher than the temperature T₃. “At least temporarily” means in the context of the present invention that this state (T₁≠T₃, preferably T₁>T₃) does not persist over the entire hydrogenation process, but is present only in or from a certain time. On the other hand, this does not mean short-term changes of state. “At least temporarily” therefore means that this state lasts for at least 30 minutes, and preferably lasts for at least 2 hours. Furthermore, “at least temporarily” may mean that at least 1% of the total process duration is characterized by this state.

This state can be realized as follows: Stream A is passed into the reactor at the temperature T₁ via the input stream (2b). Owing to the exothermicity of the hydrogenation reaction, the temperature of the reaction mixture in the reactor increases, preferably over the length of the reactor. The resulting mixture of the product stream (3) has a temperature T₂ which is higher than T₁.

A substream of the resulting mixture or of the product stream (3) having the temperature T₂ is recycled and passed with fresh reactant (2a) to stream A (2b). However, since stream A (2b) should have a lower temperature T₁ than T₂, the process must be set up such that this condition is met. It would be possible, for example, to adjust the temperature of the fresh reactant such that the temperature T₁ is established during mixing with the recycled substream. In the context of the present invention, however, it is preferable that the mixture obtained from the first hydrogenation unit or the product stream (3) is cooled down from the temperature T₂ before the mixture is separated. For this purpose, it is possible preferably to use known cooling devices. Preference is given to the use of a heat exchanger in order to utilize the extracted heat energy elsewhere in the process or in an integrated system of two or more plants.

If the mixture obtained from the first hydrogenation unit is cooled, the two substreams after the separation have equal temperature. The temperatures of T₁ and T₃ are therefore equal or differ by a maximum of 10%.

In order to achieve the state according to the invention (T₁≠T₃, preferably T₁>T₃), different measures can be taken in consideration of this procedure. Preferably, a further cooling or a heater is present on the side of one of the substreams in order to cool one substream or to heat one of the substreams. A particularly preferred method in the context of the present invention is the establishment of a bypass (4) upstream of the cooler (7) to the substream conducted to the first hydrogenation unit. By means of the bypass, a predeterminable portion of the mixture obtained from the first hydrogenation unit or of the product stream (3) can be combined uncooled with the substream (6b) to be recycled, as a result of which the temperature T₁ is greater than the temperature T₃ of the substream (6c) conducted to the second hydrogenation unit, which is passed completely via the cooler (7).

The advantage is that the hydrogenation in the first hydrogenation unit (1) can be operated at a higher entry temperature T₁, as a result of which higher hydrogenation temperatures are possible. The activity losses of the catalyst over time are thus compensated. At the same time, the reaction in the second hydrogenation unit can be operated continuously with constant temperature conditions. The total yield and the quality of the product stream thus remain constant.

In the context of the present invention, it has been found that it is advantageous when the product stream (3) having the temperature T₂ is not fed directly to the at least one further hydrogenation unit, but is instead cooled and is fed at a lower temperature T₃ to the at least one further hydrogenation unit. It is therefore particularly preferable that the temperature T₂ of the resulting mixture in step iii. is higher than the temperature T₃ of the mixture which is fed into the hydrogenation unit in step iv.

Furthermore, it has been found in the context of the present invention that it is advantageous if the temperature difference ΔT between T₂ and T₃ is constant over the temporal course of the process. Preferably, the temperature difference ΔT is equivalent to the enthalpy of reaction of the hydrogenation reaction. “Constant” in the context of the present invention means a maximum deviation of the temperature by ±10%, preferably ±5%, particularly preferably ±1%, based on the initial temperature.

The temperature regulation can be achieved by arranging, between the output stream (3) of the first hydrogenation unit and the input stream (9) of the second hydrogenation unit, an apparatus (7) which cools the output stream (3) in order to achieve the desired temperatures T₁ and T₃ in the input stream (2b) to the first hydrogenation unit and in the input stream (9) to the at least one further hydrogenation unit. Preferably, the cooling is achieved via a heat exchanger. Appropriate apparatuses are familiar to those skilled in the art. It may be present, for example, in the output stream (3) of the first hydrogenation unit (1), in the input stream (9) to the second hydrogenation unit or between the two streams. It is possible preferably to use one, two, three or more heat exchangers.

It is preferred in the context of the process according to the invention that in step i. one or more aromatic carboxylic esters, preferably one or more aromatic mono-, di- and polycarboxylic esters, are provided.

In the context of the process according to the invention, aromatic compounds such as aromatic poly- and/or monocarboxylic acids or derivatives thereof, in particular the alkyl esters thereof, can be converted to give the corresponding alicyclic polycarboxylic acid compounds. Both full esters and partial esters can be hydrogenated. A full ester is understood to be a compound in which all acid groups have been esterified. Partial esters are compounds having at least one free acid group (or possibly anhydride group) and at least one ester group.

If polycarboxylic esters are used in the process according to the invention, these preferably contain 2, 3 or 4 ester functions.

It is preferred in the context of the process according to the invention that in step i. one or more benzene-, diphenyl-, naphthalene-, diphenyl oxide-, anthracenedi- or -polycarboxylic esters are provided. The alicyclic polycarboxylic acids obtained with the process according to the invention or the derivatives thereof consist of one or more C₆ rings where appropriate linked by a carbon-carbon bond or fused.

It is further preferred that in step i. one or more aromatic carboxylic esters having an alcohol component selected from the group consisting of branched or unbranched alkoxyalkyl, cycloalkyl and/or alkyl groups having 1 to 25 carbon atoms, preferably C₈-C₁₀ phthalate, C₈-C₁₀ terephthalate, C₈-C₁₀ isophthalate and C₈-C₁₀ trimellitate, particularly preferably di-2-ethylhexyl phthalate, diisononyl phthalate, di-2-ethylhexyl terephthalate, diisononyl terephthalate, di-2-ethylhexyl isophthalate, diisononyl isophthalate, tri-2-ethylhexyl trimellitate and triisononyl trimellitate, are provided.

Here, C₈ preferably means 2-ethylhexyl or n-octyl, C₉ means isononyl and C₁₀ means isodecyl or 2-propylheptyl.

The process is preferably a process for hydrogenating 1,2-; 1,3- or 1,4-benzenedicarboxylic esters and/or 1,2,3-; 1,2,4- or 1,3,5-benzenetricarboxylic esters, i.e. the isomers of 1,2-; 1,3- or 1,4-cyclohexanedicarboxylic esters or of 1,2,3-; 1,3,5- or 1,2,4-cyclohexanetricarboxylic esters are formed.

In the process according to the invention, for example, esters of the following aromatic carboxylic acids can be used: 1,2-naphthalenedicarboxylic acid, 1,3-naphthalenedicarboxylic acid, 1,4-naphthalenedicarboxylic acid, 1,5-naphthalenedicarboxylic acid, 1,6-naphthalenedicarboxylic acid, 1,7-naphthalenedicarboxylic acid, 1,8-naphthalenedicarboxylic acid, phthalic acid (benzene-1,2-dicarboxylic acid), isophthalic acid (benzene-1,3-dicarboxylic acid), terephthalic acid (benzene-1,4-dicarboxylic acid), benzene-1,2,3-tricarboxylic acid, benzene-1,2,4-tricarboxylic acid (trimellitic acid), benzene-1,3,5-tricarboxylic acid (trimesic acid), benzene-1,2,4,5-tetracarboxylic acid (pyromellitic acid). It is also possible to use acids that are formed from the mentioned acids by substitution of one or more hydrogen atoms bonded to the aromatic ring with alkyl, cycloalkyl or alkoxyalkyl groups.

Preference is given to using alkyl, cycloalkyl and alkoxyalkyl esters for example of the abovementioned acids, where these radicals independently comprise 1 to 25, in particular 3 to 15, very particularly 8 to 13 carbon atoms, and in particular 9 carbon atoms. These radicals may be linear or branched. If a starting material has more than one ester group, these radicals may then be identical or different.

Examples of esters of an aromatic polycarboxylic acid that can be used in the process according to the invention include the following compounds: monomethyl terephthalate, dimethyl terephthalate, diethyl terephthalate, di-n-propyl terephthalate, dibutyl terephthalate, diisobutyl terephthalate, di-tert-butyl terephthalate, dipentyl terephthalate, monoglycol terephthalate, diglycol terephthalate, n-octyl terephthalate, diisooctyl terephthalate, di-2-ethylhexyl terephthalate, di-n-nonyl terephthalate, diisononyl terephthalate, di-2-propylheptyl terephthalate, di-n-decyl terephthalate, di-n-undecyl terephthalate, diisodecyl terephthalate, diisododecyl terephthalate, ditridecyl terephthalate, di-n-octadecyl terephthalate, diisooctadecyl terephthalate, di-n-eicosyl terephthalate, monocyclohexyl terephthalate; monomethyl phthalate, dimethyl phthalate, di-n-propyl phthalate, di-n-butyl phthalate, diisobutyl phthalate, di-tert-butyl phthalate, monoglycol phthalate, diglycol phthalate, di-n-octyl phthalate, diisooctyl phthalate, di-2-ethylhexyl phthalate, di-n-nonyl phthalate, diisononyl phthalate, di-n-decyl phthalate, di-2-propylheptyl phthalate, diisodecyl phthalate, di-n-undecyl phthalate, diisoundecyl phthalate, ditridecyl phthalate, di-n-octadecyl phthalate, diisooctadecyl phthalate, di-n-eicosyl phthalate, monocyclohexyl phthalate; dicyclohexyl phthalate, monomethyl isophthalate, dimethyl isophthalate, diethyl isophthalate, di-n-propyl isophthalate, di-n-butyl isophthalate, diisobutyl isophthalate, di-tert-butyl isophthalate, monoglycol isophthalate. diglycol isophthalate, di-n-octyl isophthalate, diisooctyl isophthalate, di-2-ethylhexyl isophthalate, di-n-nonyl isophthalate, diisononyl isophthalate, di-n-decyl isophthalate, diisodecyl isophthalate, di-n-undecyl isophthalate, diisododecyl isophthalate, di-n-dodecyl isophthalate, ditridecyl isophthalate, di-n-octadecyl isophthalate, diisooctadecyl isophthalate, di-n-eicosyl isophthalate, monocyclohexyl isophthalate.

The process according to the invention is also applicable in principle to benzoic acid and esters thereof. This is understood to mean not just alkyl benzoates but also benzoates of diols, such as for example glycol dibenzoate, diethylene glycol benzoate, triethylene glycol dibenzoate or propylene glycol dibenzoate. The alcohol component of the alkyl benzoates can consist of 1 to 25, preferably 8 to 13, carbon atoms and be in each case linear or branched.

On the industrial scale, aromatic esters, in particular full esters, are often preferably prepared from alcohol mixtures. Examples of appropriate alcohol mixtures include:

C₅ alcohol mixtures prepared from linear butenes by hydroformylation and subsequent hydrogenation; C₅ alcohol mixtures prepared from butene mixtures comprising linear butenes and isobutene by hydroformylation and subsequent hydrogenation; C₆ alcohol mixtures prepared from a pentene or from a mixture of two or more pentenes by hydroformylation and subsequent hydrogenation; C₇ alcohol mixtures prepared from the trimerization of ethylene or dimerization of propylene or a hexene isomer or another mixture of hexene isomers by hydroformylation and subsequent hydrogenation; C₈ alcohol mixtures, such as 2-ethylhexanol (2 isomers), prepared by aldol condensation of n-butyraldehyde and subsequent hydrogenation; C₉ alcohol mixtures prepared from C₄ olefins by dimerization, hydroformylation and hydrogenation. The preparation of the C₉ alcohols may proceed from isobutene or from a mixture of linear butenes or from mixtures of linear butenes and isobutene. The C₄ olefins may be dimerized with the aid of various catalysts, for example protic acids, zeolites, organometallic nickel compounds or solid nickel-containing catalysts. The hydroformylation of the C₈ olefin mixtures can be effected with the aid of rhodium or cobalt catalysts. There are therefore a multitude of technical C₉ alcohol mixtures; C₁₀ alcohol mixtures prepared from tripropylene by hydroformylation and subsequent hydrogenation; 2-propylheptanol (2 isomers) prepared by aldol condensation of valeraldehyde and subsequent hydrogenation; C₁₀ alcohol mixtures prepared from a mixture of at least two C₅ aldehydes by aldol condensation and subsequent hydrogenation; C₁₃ alcohol mixtures prepared from hexaethylene, tetrapropylene or tributene by hydroformylation and subsequent hydrogenation.

Other alcohol mixtures may be obtained by hydroformylation followed by hydrogenation from olefins or olefin mixtures which arise, for example, in Fischer-Tropsch syntheses, in dehydrogenations of hydrocarbons, in metathesis reactions, in the polygas process, or in other industrial processes. Olefin mixtures with olefins of differing carbon numbers may also be used to prepare alcohol mixtures.

In the process according to the invention, any ester mixture prepared from aromatic polycarboxylic acids and the abovementioned alcohol mixtures may be used. According to the invention, preference is given to using esters prepared from phthalic acid or phthalic anhydride and also terephthalic acid or dimethyl terephthalate and a mixture of isomeric alcohols having 4 to 13 carbon atoms.

Preference is given to a process for preparing one or more alicyclic compounds, comprising the steps of:
i. providing a stream A (2b), comprising one or more aromatic compounds selected from the group consisting of esters of phthalic acid, isophthalic acid, terephthalic acid and/or trimellitic acid, particularly preferably selected from the group consisting of dipentyl terephthalate, di-2-ethylhexyl terephthalate, diisononyl terephthalate, dipentyl phthalate, di-2-ethylhexyl phthalate, diisononyl phthalate, dipentyl isophthalate, di-2-ethylhexyl isophthalate, diisononyl isophthalate, tripentyl trimellitate, tri-2-ethylhexyl trimellitate, triisononyl trimellitate, or mixtures thereof, and a hydrogen-containing hydrogenation gas;
ii. feeding stream A (2b) having a temperature T₁ to a first hydrogenation unit (1) and hydrogenating the one or more aromatic compounds to give one or more corresponding alicyclic compounds;
iii. obtaining a mixture as first product stream (3) having a temperature T₂ and comprising aromatic compounds and alicyclic compounds selected from the group consisting of dialkyl cyclohexane-1,2-dicarboxylates, dialkyl cyclohexane-1,3-dicarboxylates, dialkyl cyclohexane-1,4-dicarboxylates and 1,2,4-trimellitic esters, particularly preferably selected from the group consisting of dipentyl cyclohexane-1,4-dicarboxylate, di-2-ethylhexyl cyclohexane-1,4-dicarboxylate, diisononyl cyclohexane-1,4-dicarboxylate, dipentyl cyclohexane-1,2-dicarboxylate, di-2-ethylhexyl cyclohexane-1,2-dicarboxylate, diisononyl cyclohexane-1,2-dicarboxylate, dipentyl cyclohexane-1,3-dicarboxylate, di-2-ethylhexyl cyclohexane-1,3-dicarboxylate, diisononyl cyclohexane-1,3-dicarboxylate, tripentyl cyclohexane-1,2,4-tricarboxylate, tri-2-ethylhexyl cyclohexane-1,2,4-tricarboxylate, triisononyl cyclohexane-1,2,4-tricarboxylate;
iv. separating the product stream (3) obtained in step iii. into a substream (8), which is fed to stream A (2b) in step i. and hydrogenated again as part of stream A (2b) in step ii., and a substream (6c), which is fed as stream B (9) at a temperature T₃ into one or more further hydrogenation units (11);
v. hydrogenating the aromatic compounds present in stream B (9) as defined in step i. to give the corresponding alicyclic compounds in the one or more further hydrogenation units (11), and
vi. obtaining a product stream (12) at a temperature T₄ and comprising one or more alicyclic compounds selected from the group consisting of dialkyl cyclohexane-1,2-dicarboxylates, dialkyl cyclohexane-1,3-dicarboxylates, dialkyl cyclohexane-1,4-dicarboxylates and 1,2,4-trimellitic esters, particularly preferably selected from the group consisting of dipentyl cyclohexane-1,4-dicarboxylate, di-2-ethylhexyl cyclohexane-1,4-dicarboxylate, diisononyl cyclohexane-1,4-dicarboxylate, dipentyl cyclohexane-1,2-dicarboxylate, di-2-ethylhexyl cyclohexane-1,2-dicarboxylate, diisononyl cyclohexane-1,2-dicarboxylate, dipentyl cyclohexane-1,3-dicarboxylate, di-2-ethylhexyl cyclohexane-1,3-dicarboxylate, diisononyl cyclohexane-1,3-dicarboxylate, tripentyl cyclohexane-1,2,4-tricarboxylate, tri-2-ethylhexyl cyclohexane-1,2,4-tricarboxylate, triisononyl cyclohexane-1,2,4-tricarboxylate, corresponding to the one or more aromatic compounds provided in step i.,
   wherein at least temporarily the temperature T₁ is unequal to the temperature T₃, preferably wherein the temperature T₁ is higher than the temperature T₃.

The alicyclic compounds present in the product stream (3) depend on the starting material used. For example, diisononyl cyclohexane-1,2-dicarboxylate is obtained as product when using diisononyl phthalate as starting material.

Particular preference is given to a process for preparing one or more alicyclic compounds, comprising the steps of:
i. providing a stream A (2b) containing diisononyl phthalate (DINP) or di-2-ethylhexyl phthalate (DEHP) and a hydrogen-containing hydrogenation gas;
ii. feeding stream A (2b) having a temperature T₁ to a first hydrogenation unit (1) and hydrogenating the one or more aromatic compounds to give one or more corresponding alicyclic compounds;
iii. obtaining a mixture as first product stream (3) having a temperature T₂ and comprising diisononyl cyclohexane-1,2-dicarboxylate (DINCH) or di-2-ethylhexyl cyclohexane-1,2-dicarboxylate (DEHCH);
iv. separating the product stream (3) obtained in step iii. into a substream (8), which is fed to stream A (2b) in step i. and hydrogenated again as part of stream A (2b) in step ii., and a substream (6c), which is fed as stream B (9) at a temperature T₃ into one or more further hydrogenation units (11);
v. hydrogenating the aromatic compounds present in stream B (9) to give the corresponding alicyclic compounds in the one or more further hydrogenation units (11), and
vi. Obtaining a product stream (12) having a temperature T₄ and comprising diisononyl 1,2-cyclohexanedicarboxylate (DINCH) or di-2-ethylhexyl 1,2-cyclohexanedicarboxylate (DEHCH),
   wherein at least temporarily the temperature T₁ is unequal to the temperature T₃, preferably wherein the temperature T₁ is higher than the temperature T₃.

Particular preference is further given to a process for preparing one or more alicyclic compounds, comprising the steps of:
i. providing a stream A (2b) comprising diisononyl terephthalate or di-2-ethylhexyl terephthalate and a hydrogen-containing hydrogenation gas;
ii. feeding stream A (2b) having a temperature T₁ to a first hydrogenation unit (1) and hydrogenating the one or more aromatic compounds to give one or more corresponding alicyclic compounds;
iii. obtaining a mixture as first product stream (3) having a temperature T₂ comprising diisononyl 1,4-cyclohexanedicarboxylate or di-2-ethylhexyl 1,4-cyclohexanedicarboxylate;
iv. separating the product stream (3) obtained in step iii. into a substream (8), which is fed to stream A (2b) in step i. and hydrogenated again as part of stream A (2b) in step ii., and a substream (6c), which is fed as stream B (9) at a temperature T₃ into one or more further hydrogenation units (11);
v. hydrogenating the aromatic compounds present in stream B (9) to give the corresponding alicyclic compounds in the one or more further hydrogenation units (11), and
vi. Obtaining a product stream (12) having a temperature T₄ comprising diisononyl 1,4-cyclohexanedicarboxylate or di-2-ethylhexyl 1,4-cyclohexanedicarboxylate,
   wherein at least temporarily the temperature T₁ is unequal to the temperature T₃, preferably wherein the temperature T₁ is higher than the temperature T₃.

Particular preference is further given to a process for preparing one or more alicyclic compounds, comprising the steps of:
i. providing a stream A (2b) containing triisononyl trimellitate (TINTM) or tri-2-ethylhexyl trimellitate (TOTM) and a hydrogen-containing hydrogenation gas;
ii. feeding stream A (2b) having a temperature T₁ to a first hydrogenation unit (1) and hydrogenating the one or more aromatic compounds to give one or more corresponding alicyclic compounds;
iii. Obtaining a mixture as first product stream (3) having a temperature T₂ comprising triisononyl 1,2,4-cyclohexanetricarboxylate or tri-2-ethylhexyl 1,2,4-cyclohexanetricarboxylate;
iv. separating the product stream (3) obtained in step iii. into a substream (8), which is fed to stream A (2b) in step i. and hydrogenated again as part of stream A (2b) in step ii., and a substream (6c), which is fed as stream B (9) at a temperature T₃ into one or more further hydrogenation units (11);
v. hydrogenating the aromatic compounds present in stream B (9) to give the corresponding alicyclic compounds in the one or more further hydrogenation units (11), and
vi. obtaining a product stream (12) having a temperature T₄ and comprising triisononyl cyclohexane-1,2,4-tricarboxylate or tri-2-ethylhexyl cyclohexane-1,2,4-tricarboxylate;
   wherein at least temporarily the temperature T₁ is unequal to the temperature T₃, preferably wherein the temperature T₁ is higher than the temperature T₃.

The process according to the invention is preferably conducted under the following conditions.

In the feed of the first hydrogenation unit (loop mode), the concentration of the aromatic compounds as starting material is between 5% and 30% by mass, in particular between 8% and 15% by mass. In the starting stream (3) of the first hydrogenation unit, the concentration of the reactant is between 0.3% and 8% by mass, especially between 1,5% and 4% by mass. The specific liquid hourly space velocity (LHSV, litres of fresh reactant per litre of catalyst per hour) in the first hydrogenation unit (1) is 0.1 to 5 h⁻¹, in particular 0.5 to 3 h⁻¹.

The surface area velocity in the first hydrogenation unit (1) is in the range from 25 to 140 m³/m²/h, in particular in the range from 50 to 90 m³/m²/h.

The average hydrogenation temperatures in the first hydrogenation unit (1) are 70 to 150° C., in particular 80 to 120° C.

The hydrogenation pressure in the first hydrogenation unit (1) is 25 to 200 bar, in particular 80 to 110 bar.

The specific catalyst hourly space velocity of the second hydrogenation unit (11) (litres of reactant per litre of catalyst per hour) is 1 to 8 h⁻¹, in particular 2 to 5 h⁻¹.

In the second hydrogenation unit (11), the average temperature is between 70 and 150° C., in particular 80 and 120° C.

The hydrogenation pressure in the second hydrogenation unit is 25 to 200 bar, in particular 80 to 100 bar.

The process variants are suitable in particular for the hydrogenation of phthalic esters, particularly for diisononyl phthalates (as isomer mixture “diisononyl phthalate”, for example VESTINOL 9 from OXENO GmbH) or di-2-ethylhexyl phthalate.

A further aspect of the present invention is the provision of an apparatus for carrying out a process according to the invention, comprising a first hydrogenation unit (1) and one or more further hydrogenation units (11) and one or more heat exchangers (7), wherein the heat exchanger (7) is arranged such that the output stream (3) is passed via an input stream (5) into the heat exchanger (7) and the output stream of the heat exchanger (6a) has a lower temperature than the input stream (5) and is then fed via pipelines (6b, 6c) as input stream (9) into a further hydrogenation unit (11) and/or is fed via stream (10) into in turn one or more further hydrogenation units (11) and/or is fed via stream 8 to the input stream to the first hydrogenation unit (1), preferably wherein streams 3 and 9 have a different temperature.

Preferably, at least one further heat exchanger may be present, which is arranged, for example, in the input stream (9) of the at least one further hydrogenation unit (11).

It is preferable that, in the context of the apparatus according to the invention, the first and/or at least one of the further hydrogenation units (1,11) comprises one or more fixed-bed catalysts, preferably wherein the solid catalyst comprises at least one metal of the eighth transition group of the periodic table of the elements, particularly preferably ruthenium. The preferred support material is titanium dioxide. What has been stated herein for the catalysts used applies correspondingly.

It is likewise preferable that the pipelines of the first hydrogenation unit (1) are arranged such that the output stream (3) of the first hydrogenation unit (1) can be recycled via a bypass (4) or via the pipelines (6b, 8) as input stream (2b) to the first hydrogenation unit (1). The first hydrogenation unit (1) is then operated in loop mode.

It is further preferred that in the first and/or in one of the further hydrogenation units there is a mixture present of aromatic compounds and corresponding alicyclic compounds, preferably a mixture of aromatic carboxylic esters and corresponding alicyclic compounds thereof having an alcohol component selected from the group consisting of branched or unbranched alkoxyalkyl, cycloalkyl and/or alkyl groups having 1 to 25 carbon atoms, preferably from C₈-C₁₀ phthalate, C₈-C₁₀ terephthalate, C₈-C₁₀ isophthalate and C₈-C₁₀ trimellitate, particularly preferably di-2-ethylhexyl phthalate, diisononyl phthalate, di-2-ethylhexyl terephthalate, diisononyl terephthalate, di-2-ethylhexyl isophthalate, triisononyl isophthalate, di-2-ethylhexyl trimellitate and triisononyl trimellitate, diisononyl phthalate and/or didecyl phthalate and diisononyl cyclohexanedicarboxylates and/or didecyl cyclohexanedicarboxylates and the corresponding alicyclic compounds thereof. What has been stated herein for the starting materials (aromatic compounds) and products (alicyclic compounds) applies correspondingly.

Preference in the context of the present invention is given to the use of the alicyclic polycarboxylic esters prepared according to the invention as plasticizers in plastics. Preferred plastics are PVC, homo- and copolymers based on ethylene, propylene, butadiene, vinyl acetate, glycidyl acrylate, glycidyl methacrylate, acrylates, acrylates with alkyl radicals, bonded to the oxygen atom of the ester group, of branched or unbranched alcohols having one to ten carbon atom(s), styrene, acrylonitrile or homo- or copolymers of cyclic olefins.

In addition to the abovementioned applications, the alicyclic polycarboxylic esters prepared according to the invention can be used as a lubricating oil component, as a constituent of cooling liquids and metal machining liquids. They can also be used as a component in paints, coatings, inks and adhesives.

The examples which follow are intended to illustrate the invention without restricting its scope of application resulting from the description.

### EXAMPLES

The examples which follow describe the performance of the hydrogenation of diisononyl phthalate (DINP) to give diisononyl 1,2-cyclohexanedicarboxylate (DINCH), the intention being to attain a final concentration of DINP in the output from the last stage of less than 0.05% by weight of the input concentration in the 1st stage.

All examples were simulated by means of the Aspen V10 software on the basis of real process data and kinetic models based thereon. The hydrogenation unit consisted of a reactor which was operated continuously in loop mode and a subsequent reactor which was operated in straight pass mode. In all experiments, the liquid phase and the hydrogenation gas flow in cocurrent from top to bottom.

In accordance with FIG. 1, the entry temperature of the first reactor is referred to as T₁ (2b), the exit temperature of the second reactor as T₂ (3), the entry temperature of the third reactor as T₃ (9), and the exit temperature of the second reactor as T₄ (12).

If the reaction is started and a catalyst activity of 100% and full plant load are assumed, the following temperatures result:

100% Catalyst activity, 100% Plant load

| T₁/° C. | T₂/° C. | T₃/° C. | T₄/° C. |
|---|---|---|---|
| 101.7 | 115.5 | 107.4 | 140.7 |

Over time, the activity of the catalyst decreases. If a catalyst activity of 70% and full plant load are assumed, the following temperatures result:

70% Catalyst activity, 100% Plant load

| T₁/° C. | T₂/° C. | T₃/° C. | T₄/° C. |
|---|---|---|---|
| 104.5 | 117.6 | 110.4 | 152.6 |

The temperature at the entry must be increased in order to be able to still achieve the final concentration of DINP of less than 0.05% by weight.

### Example 1 (Comparative Example)

As the time progresses, the activity of the catalyst decreases further. If a catalyst activity of 30% and full plant load are assumed, the following temperatures result:

30% Catalyst activity, 100% Plant load

| T₁/° C. | T₂/° C. | T₃/° C. | T₄/° C. |
|---|---|---|---|
| 122.3 | 134.8 | 125.4 | 172.0 |

In order to be able to maintain the final concentration of DINP of less than 0.05% by weight, the entry temperature T₁ must be increased further. Owing to the heat of reaction formed, the temperature T₄ rises to such an extent that the plant climbs above the maximum permitted temperature of 160° C. The plant has to be shut down. Another possibility would be a reduction in plant load, which, however, would result in considerably lower production rates.

### Example 2 (Inventive Example)

Example 2 was carried out with the temperature regulation according to the invention, in which T₁ and T₃ can be set independently of one another and temperature T₁ is unequal to the temperature T₃. If a catalyst activity of 30% and full plant load are assumed, the following temperatures result:

30% Catalyst activity, 100% Plant load

| T₁/° C. | T₂/° C. | T₃/° C. | T₄/° C. |
|---|---|---|---|
| 122.3 | 134.8 | 108.1 | 155.2 |

It is found that the temperature is within an acceptable range and neither is a plant shutdown imminent nor does the plant load have to be reduced.

1. A process for preparing one or more alicyclic compounds, the process comprising:
i. providing a stream A comprising one or more aromatic compounds and a hydrogen-containing hydrogenation gas;
ii. feeding stream A having a temperature T₁ to a first hydrogenation unit and hydrogenating the one or more aromatic compounds to give one or more corresponding alicyclic compounds;
iii. obtaining a mixture as first product stream having a temperature T₂ comprising one or more aromatic compounds and alicyclic compounds;
iv. separating the first product stream obtained in iii. into a first substream, which is fed to stream A in i. and hydrogenated again as part of stream A in ii., and a second substream, which is fed as stream B at a temperature T₃ into one or more further hydrogenation units;
v. hydrogenating the one or more aromatic compounds present in stream B to give corresponding alicyclic compounds in the one or more further hydrogenation units, and
vi. obtaining a second product stream having a temperature T₄ comprising one or more alicyclic compounds corresponding to the one or more aromatic compounds provided in i.,
wherein at least temporarily the temperature T₁ is unequal to the temperature T₃.

2. The process according to claim 1, comprising:
wherein the hydrogenation of the one or more aromatic compounds provided in i. is effected with the hydrogen-containing gas provided in i. over one or more solid catalysts arranged in a fixed bed of the first hydrogenation unit and/or the one or more further hydrogenation units.

3. The process according to claim 2, wherein the one or more solid catalysts comprise at least one metal from the eighth transition group of the periodic table of the elements.

4. The process according to claim 1, comprising:
carrying out iv. with a circulation ratio of 1:10 to 1:50.

5. The process according to claim 1, comprising:
carrying out the hydrogenation in ii. and/or v. at a pressure of 3 to 300 bar.

6. The process according to claim 1, comprising:
carrying out the hydrogenation in ii. and/or v. at a temperature of 50° C. to 250° C.

7. The process according to claim 1, wherein the temperature T₂ of the obtained mixture in iii. is higher than the temperature T₃ of the mixture which is supplied to the hydrogenation unit in iv.

8. The process according to claim 7, wherein a temperature difference ΔT between T₂ and T₃ is constant over a course of time of carrying out the process.

9. The process according to claim 1, comprising:
providing in i. one or more aromatic carboxylic esters.

10. The process according to claim 1, comprising:
providing in i. one or more benzene-, diphenyl-, naphthalene-, diphenyl oxide-, anthracenedi- or polycarboxylic esters.

11. The process according to claim 1, comprising:
providing in i. one or more aromatic carboxylic esters having an alcohol component selected from the group consisting of branched or unbranched alkoxyalkyl, cycloalkyl and/or alkyl groups having 1 to 25 carbon atoms, and mixtures thereof.

12. An apparatus for carrying out a process according to claim 1, the apparatus comprising:
a first hydrogenation unit,
one or more further hydrogenation units, and
one or more heat exchangers,
wherein the one or more heat exchangers are arranged that the first product stream is passed via a first input stream into the one or more heat exchangers and an output stream of the one or more heat exchangers has a lower temperature than the input stream and is then fed via one or more pipelines as a second input stream into one or more further hydrogenation units and/or is fed via stream into in turn one or more further hydrogenation units and/or is fed via stream to an input stream to the first hydrogenation unit.

13. The apparatus according to claim 12, wherein the first hydrogenation unit and/or one of the one or more further hydrogenation units has one or more fixed-bed catalysts.

14. The apparatus according to claim 12, wherein the one or more pipelines of the first hydrogenation unit are arranged that the first product stream of the first hydrogenation unit is configured to be passed via a bypass or via the one or more pipelines as stream A to the first hydrogenation unit.

15. The apparatus according to claim 12, wherein, in the first and/or in one of the one or more further hydrogenation units, a mixture of aromatic compounds and corresponding alicyclic compounds is present.
